(19) United States
(12) Patent Application Publication
(10) Pub. No.: US 2021/0346208 A1
(43) Pub. Date: Nov. 11, 2021

(54) AlgaeAid

(71) Applicants: Meghana Sai Sunkara, Las Vegas, NV (US); Jang Choe, Henderson, NV (US); Darren Zhong, Las Vegas, NV (US)

(72) Inventors: Jang Choe, Las Vegas, NV (US); Darren Zhong, Las Vegas, NV (US); Meghana Sai Sunkara, Las Vegas, NV (US)

(73) Assignee: Meghana Sai Sunkara, Las Vegas, NV (US)

(21) Appl. No.: 17/141,009

(22) Filed: Jan. 4, 2021

Related U.S. Application Data
(60) Provisional application No. 63/020,357, filed on May 5, 2020.

(51) Int. Cl. A61F 13/02; A61F 13/00; A61L 26/00

(52) U.S. Cl. CPC A61F 13/0213; A61F 13/00063; A61L 26/008; A61L 2300/402; A61L 26/0052; A61L 26/0066; A61L 2300/406; A61L 26/0023

(57) ABSTRACT

AlgaeAid is an inulin-integrating hydrogel dressing aimed at the treatment of second and third-degree burns and infected chronic wounds, which lack an optimal dressing. Currently, the vast majority of dressings for burns and chronic wounds are not optimal for all kinds of wounds. AlgaeAid's implementation of inulin separates it from the rest of the antibiotic hydrogels. The inulin allows for increased water retention within a hydrogel because it becomes osmotically active when combined with a liquid. As this property allows plants to remain hydrated and withstand dry and cold temperatures, increasing water retention. The flexibility that inulin provides makes AlgaeAid more universally applicable and allows for the treatment of wounds with more fluid expulsion which makes it effective against superbugs such as MRSA and can be readily sourced from a wide variety of algae and plant sources, including chicory, algae, and 35 other plant species.

### CROSS-REFERENCE TO RELATED APPLICATIONS

Provisional Application No. 63/020,357

### STATEMENT REGARDING FEDERALLY SPONSORED RESEARCH OR DEVELOPMENT

### Incorporation-by-Reference of Material Submitted on a Compact Disc or as a Text File Via the Office Electronic Filing System (EFS-Web)

### Statement Regarding Prior Disclosures by the Inventor or a Joint Inventor

### BACKGROUND OF THE INVENTION

### (1) Field of the Invention

Medicinal Hydrogels

### (2) Description of Related Art Including Information Disclosed Under 37 CFR 1.97 and 1.98

The wound dressing market for burns, which is growing at an astounding 8.6% annually, is dominated by colloidal silver, hydrogel, calcium alginate, and hydrocolloid bandages, each with their own flaws. Although colloidal silver bandages are commonly used for chronic wounds and colloidal silver is a proven antibiotic, the bandage has its own flaws that may detriment its users in the long term. Structurally, most colloidal silver bandages are marketed as the conventional adhesive and cotton bandage with the cotton pad replaced by one soaked in colloidal silver, limiting its use to minor cuts and injuries and unable to be implemented in diverse settings. 50% of the population have sensitivities to the epoxy-based adhesives used to adhere the cotton pad to the skin, so colloidal silver bandages are not optimal for a large portion of the population. The dressings used for burns are often unable to be implemented with other antibiotics as not only is colloidal silver hampered by the use of other antibiotic ointments, but it may negatively react with antibiotics such as penicillin, amoxicillin, and tetracycline. However, most concerningly, is the adverse impacts colloidal silver has on the immune system. Long term exposure on open wounds causes colloidal silver to build up in the body leading to agrygia, a discoloration of the body to a blue-gray tone and increases an individual's risk for seizures and kidney failure as silver may start to attack the body's cells. Additionally, materials, like colloidal silver, which are expensive to implement even in small quantities, have hampered the growth of the market. In that sense, AlgaeAid's use of inexpensive and natural algae derivatives allows for a bulk of AlgaeAid products to be created without a financial burden.

Hydrogel bandages and calcium alginate bandages are another predominant dressing used for burn victims in the medical field today. Hydrogel bandages are widely praised as an effective bandaid with natural antibiotic and adhesive properties. They utilize a hypoallergenic substance composed of 90% water that exerts negative pressure. This ensures the consistent delivery of moisture into the wound while filling up any gaps in the skin to prevent biofilm development and speed up regeneration. Despite these benefits and the current widespread use of hydrogel bandages in wound treatment, hydrogels can't be used in wounds that emit pus as it renders hydrogels useless as water fails to be retained. This limits the versatility of hydrogel bandages and calls for repeated dressing replacement, which increases the risk of infection. Calcium alginate dressings are also used for similar purposes and have many of the same antibiotic properties and naturally adhesive properties that hydrogels also have. The only difference is that they occupy a different niche in the market. As calcium alginate dressings utilize a highly polymerized dressing using the natural algae-derived antibiotic calcium alginate, it can be used for extremely large wounds that emit a large amount of liquid. Unfortunately, the extensive polymerization process that is required in the manufacturing of this product often puts calcium alginate dressings on the pricier side. Additionally, its bulk prevents its use in wounds that do not emit a large amount of liquid as it requires moisture to function properly. This leaves a gap in the market for a dressing that has a more universal function that can address wounds that aren't large enough or bulky enough for calcium alginate dressings yet have too much fluid for hydrogel dressings. This is where AlgaeAid comes in.

After our team compared colloidal silver, hydrogels, and two algae-derived antibiotics (inulin and beta-glucan) over the course of two weeks, we discovered that inulin is the most effective against bacteria, such as Staphylococcus aureus and P. acnes, normally present in the skin's microflora. Inulin, a compound naturally found in algae, chicory roots, fruits, wheat, vegetables, and herbs, can increase water retention due to its nonpolar structural moieties, is resistant to degradation by enzymes in the skin, and increases the mesh structure of hydrogel giving it better mechanical strength. Thus, AlgaeAid is a product that merges the properties of inulin with that of hydrogels to address flaws in current dressings and provide a product that not only fills the gap in the wound dressing market, but has a more universal function to treat all wounds regardless of the size or severity of fluid loss.

### BRIEF SUMMARY OF THE INVENTION

AlgaeAid is a hydrogel wound dressing that incorporates inulin, a fructooligosaccharide with antibiotic properties

### BRIEF DESCRIPTION OF THE SEVERAL VIEWS OF THE DRAWING(S)

FIG. 1 shows Three bacterial plates containing three different types of algae derivatives (Inulin (the optimal one), beta-glucan, and colloidal silver) with hydrogel dressing

FIG. 2 shows Three algae derivative bacterial plates after 24 hours of bacterial growth

FIG. 3 shows Three algae derivative based bacterial plates after 3 days of bacterial growth

FIG. 4 shows Three algae derivative based bacterial plates after 5 days of bacterial growth

FIG. 5 shows Hydrogel dressing prep by soaking the bandage in water before placing in bacterial plates

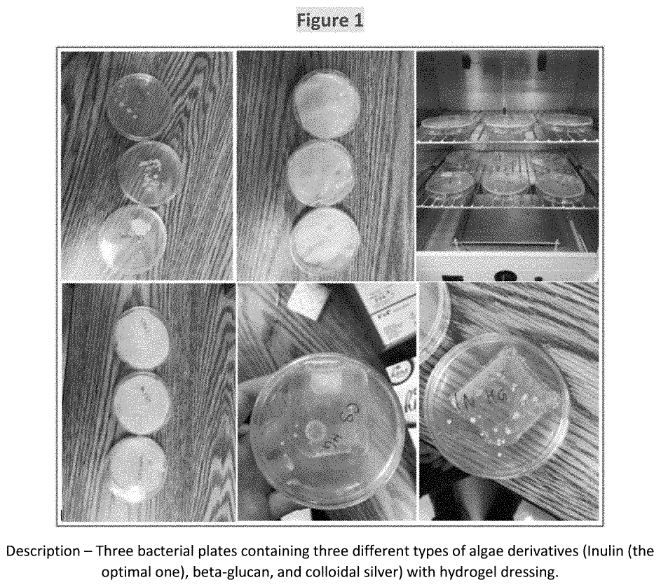

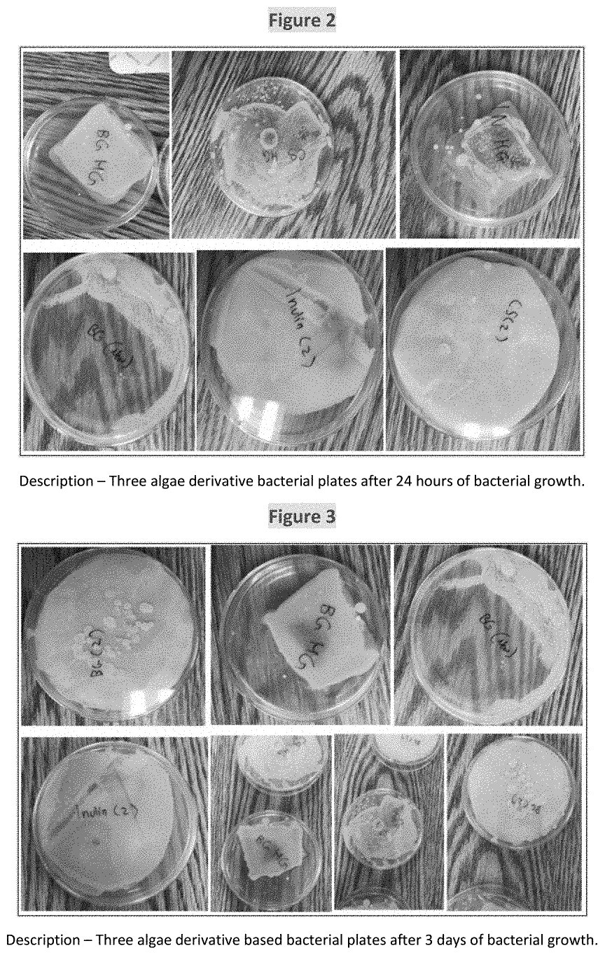

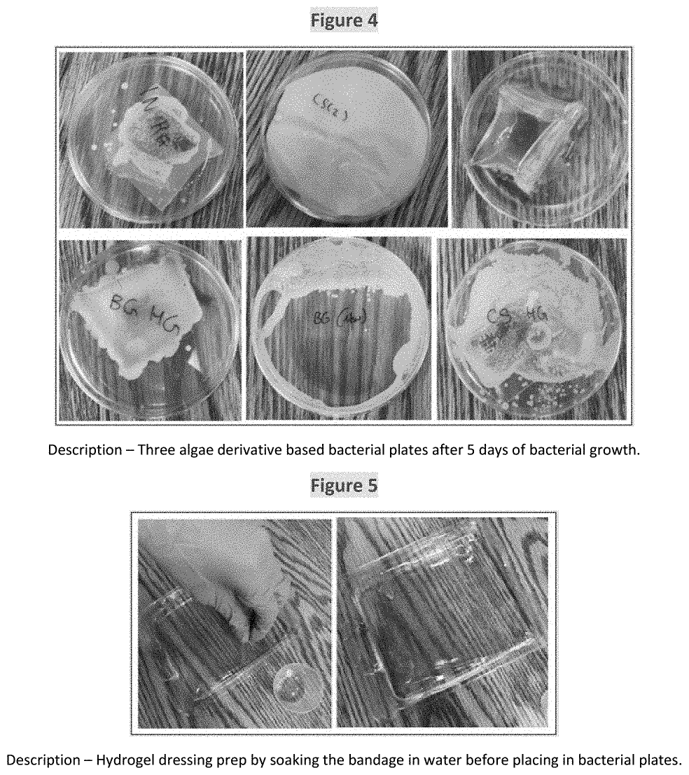

### DETAILED DESCRIPTION OF THE INVENTION

AlgaeAid utilizes two technical and scientific principles in order to maintain design and maximize functionality. The first is the concept of hydrogel wound dressings, a polymerized gel substrate composed of 90% water. AlgaeAid utilizes hydrogel technology and added structural modifications in order to address the current flaws of hydrogels while maintaining the positives that have allowed for hydrogel's success.

The first component, hydrogel's gel substrate, is responsible for hydrogel's antibiotic nature and natural adhesive properties. Hydrogel's natural adhesive properties make it a great alternative to traditional bandages that use epoxy resins which 50% of the population has a sensitivity to. The polymerization process commonly used in the manufacture of hydrogels accommodates solutions with water-soluble antibiotics. This allows for the suspension of antibiotics in water and, consequently, the consistent administration of antibiotics to the wound. The mechanical structure of hydrogel also allows for the absorption of bodily fluids that are expelled from the wound until the hydrogel's water has been largely displaced by the incoming bodily fluids. In addition, greater re-epithelialization, enhanced extracellular matrix remodeling, and greater nerve reinnervation are all benefits to wound healing that have come from the use of a gel substrate dressing.

The second component, the liquid solution, keeps the wound hydrated, which is imperative when considering that most burn wound complications are a result of dehydration or infection. Currently, most hydrogels only utilize water in their makeup and do not use water-soluble antibiotics despite the ease at which they can be incorporated. Antibiotics such as penicillin, tetracycline, and amoxicillin are not only expensive, but superbugs commonly found in infected wounds such as Methicillin-Resistant Staphylococcus aureus (MRSA) have developed resistance to such water-soluble antibiotics. However, the hydration that hydrogels provide often suffices to prevent the growth of biofilms, communities of bacteria that commonly form on untreated wounds and have antibiotic-resistant properties, as they cannot grow in the presence of moisture. Although this has led to the success of hydrogels in the market, recent studies have revealed that bugs such as MRSA can still infect wounds using the currently available water-only hydrogels. Unfortunately, this 90% water structure has led to another flaw: hydrogels cannot accommodate large amounts of fluid being released from wounds. This not only limits the versatility of the dressing, but results in a need for frequent bandage replacement, increasing the risk of infection.

Thus, we have implemented a second technical principle, algae-derived antibiotics, in order to complement the hydrogel gel substrate. Due to superbugs such as MRSA being resistant to a large number of commonly available antibiotics, we had to search for an alternative antibiotic that common bacteria had not yet been widely exposed to yet. After coming across calcium alginate bandages that utilize sodium alginate, an algae-derived antibiotic, we were inspired to test several algae-derived antibiotics that were inexpensive and easy to harvest. After reducing our list of antibiotics to two, we compared our chosen antibiotics, inulin and beta-glucan, against colloidal silver and a standard hydrogel. After incubating epithelial bacteria and applying our chosen antibiotics to our colonies, we discovered that inulin was the most effective against colonies of epithelial bacteria.

Inulin, a water-soluble fructooligosaccharide, is found most commonly in perennial chicory roots which are able to be grown throughout Europe and North America. It is also found in a variety of other algae and plants including chlorella, agave, burdock, banana, camas, coneflower, costus, dandelion, elecampane, garlic, globe artichoke, Jerusalem artichoke, jicama, leopard's bane, mugwort, onion, wild yam, and yacon. Inulin is also synthesizable in laboratories from sucrose. Today, it is commonly used as an FDA approved nutritional supplement as a “diabetic sugar”, but its chemical structure has proven to yield a plethora of additional benefits beyond that of nutritional value. Inulin has multiple glucosyl and fructosyl moieties which allow for β2-1 linkages to form during polymerization. This polymerization process is relatively simple as when inulin is mixed with a liquid, it easily forms a gel network that is osmotically active. Due to this property, by changing the amount of polymerization, one can immobilize large amounts of water and improve the stability of polymers such as foams and emulsions. In the natural world, plants change the polymerization of inulin during the winter to regulate cold resistance and store energy in the winter and these moieties prevent the digestion of inulin by human enzymes amylase and ptyalin.

In practical application, these properties are optimal to address the flaws of current hydrogel technology. Inulin would increase the water retention of hydrogels, reduce turnover rates for reapplication of dressings, stabilize the current gel substrate structure, and provide a natural algae-derived antibiotic that superbugs such as MRSA are not resistant to and are easy to harvest. The simple polymerization process that can be adjusted to fit the needs of hydrogels would complement the current hydrogel technology if a solution of inulin and water were to replace the current water-only solution that makes up 90% of hydrogels. Additionally, the inability of human enzymes to digest inulin would ensure consistent delivery of the antibiotic to the wound without degradation over time making the fluid capacity of the dressing the only factor to determine the turnover time for dressing reapplication.

1. A hydrogel dressing for treating burns and chronic wounds that incorporates inulin in any form whether in the form of a powder, suspended in the hydrogel, or integrated into the gel polymer substrate, said hydrogel comprising:
A. Backing material
B. Adhesive
C. Inulin
D. Hydrogel substrate

2. The wound dressing of claim 1, wherein the gel polymer has an adhesive on at least one side.

3. The wound dressing of claim 1, wherein the netting member includes a transparent material.

4. The wound dressing of claim 1, wherein the fluid is selected from the group consisting of: a gel, lotion, adhesive, cyanoacrylate, polyacrylate, tissue sealant, cream, ointment, fibrin, fibrin-like substance, medicament, anesthetic, antibiotic, wound healing agent, and combinations thereof.

5. The wound dressing of claim 1, wherein the gel dressing includes a polymer structure.

6. The wound dressing of claim 1, wherein the gel dressing is adaptable for contouring to the patient's skin.

7. The wound dressing according to claim 1, wherein said hydrogel is in sheet form.

8. The wound dressing of claim 1, wherein said backing material is breathable.

9. The wound dressing of claim 1, wherein said backing material and said adhesive are breathable.

10. The wound dressing of claim 1, wherein bodily fluids can be absorbed.

11. The wound dressing of claim 1, wherein its gel substrate is created using a polymerization process that accommodates solutions with water-soluble antibiotics.

12. The wound dressing of claim 1, wherein the polymerization process utilizes an inulin-water solution of any concentration as the liquid for the polymer.
